(19) United States
(12) Patent Application Publication
(10) Pub. No.: US 2012/0067749 A1
(43) Pub. Date: Mar. 22, 2012

(54) Container

(76) Inventor: Christine Evelyn Coombes, Eymet (FR)

(21) Appl. No.: 13/262,585

(22) PCT Filed: Apr. 1, 2010
(86) PCT No.: PCT/GB10/00654

§ 371 Date: Dec. 9, 2011

(30) Foreign Application Priority Data
Apr. 3, 2009 (GB) 0905847.0

(51) Int. Cl. B65D 81/24; B65D 25/04; B65B 55/18

(52) U.S. Cl. 206/205; 53/431; 220/500

(57) ABSTRACT

A container (10) with removable end caps (12, 14) has an internal barrier (30) with holes (32) therethrough. In use, a cleaning or other fluid is poured into lower space (38) and an article to be cleaned or otherwise treated is placed into upper space (36). End cap (12) is then secured and the container is inverted so that the article is in the liquid. To remove the article, which may be a cloth or a sponge, the container (10) is restored to its original disposition, end cap (12) is removed and the article is squeezed by the user against the barrier (30) to remove excess liquid.

The present invention relates to a container and in particular to a container for damp articles, especially for sterilising and storing articles.

A problem which arises in kitchens is the storage of dish cloths, sponges, and scouring pads and the like. These articles have been found to harbour high levels of bacteria around the home. If they are left on a draining board or placed next to a tap, they look unsightly. If they are stored in a cupboard, there results an unpleasant smell. If they are placed in a jar or other conventional container to produce a tidy effect, there are still the problems of smell and hygiene.

Aspects of the present invention seek to overcome or at least reduce one or more of the above problems.

According to a first aspect of the present invention, there is provided a method of accommodating an article at least partly under liquid employing a closeable container having an interior space which is divided into two by a permeable barrier, the method comprising the steps of providing a liquid in a space below the barrier, inserting an article into a space above the barrier, closing the container and then inverting the container.

If an article is stored in liquid located below the barrier, inversion of the container removes the article from the liquid, so that access is possible to the article without it being any longer immersed in the liquid. Thus the liquid and the article can be mixed or separated at will. The storage of the article in liquid is hygienic and avoids unpleasant smells. It is also an attractive arrangement. The access to the article without it being immersed in liquid avoids waste of liquid; the liquid is conveniently retained in the container for subsequent re-use to store the article again.

The article is preferably a dish cloth or other piece of kitchen equipment.

According to a second aspect of the present invention, there is provided a closeable container which, apart from a removable cap for opening and closing the container at one or both ends, is of integral construction, the container having an interior space which is divided into two by an integral permeable barrier spaced from both ends.

According to a third aspect of the present invention, there is provided a container comprising a housing which, apart from a removable cap for opening and closing the container at one or both ends, is of integral construction, the housing having an interior space which is divided into two by a permeable barrier which is attached to the housing substantially half way between the ends.

According to a fourth aspect of the present invention, there is provided a closeable container having an interior space which is divided into two by a permeable barrier, the space below the barrier containing a sterilising liquid.

A preferred embodiment of the present invention will now be described, by way of example only, with reference to the accompanying drawings, of which:

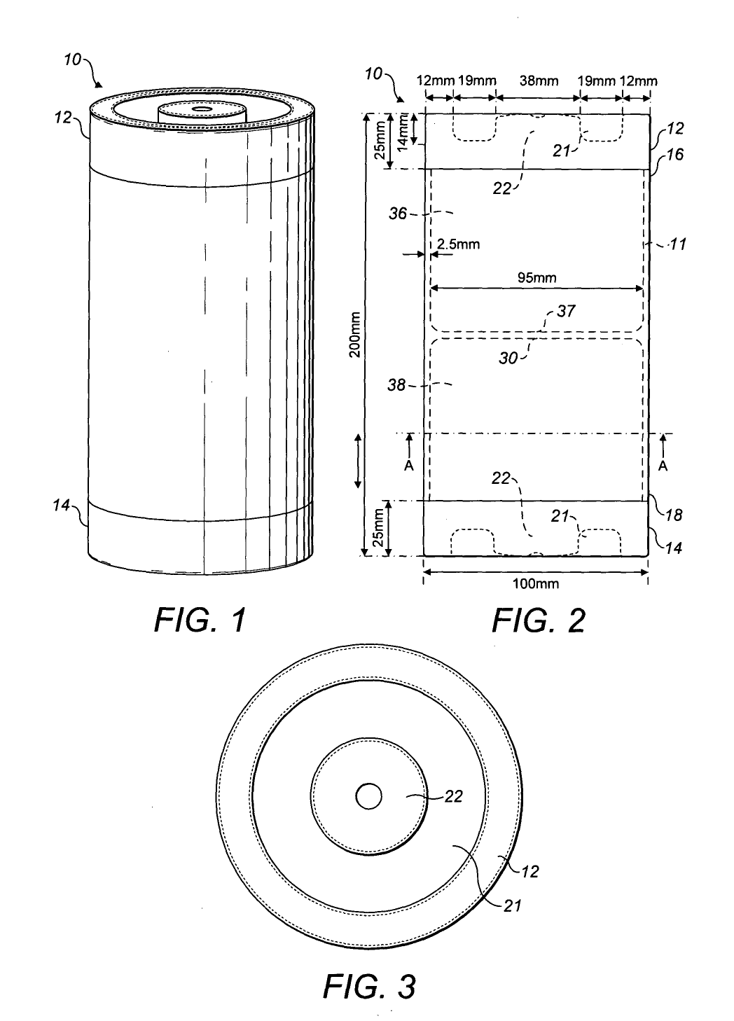

FIG. 1 is a perspective view of a container in accordance with the present invention;

FIG. 2 is a side view of the container of FIG. 1, with preferred dimensions being indicated in mm;

FIG. 3 is an end view of the container of FIG. 1;

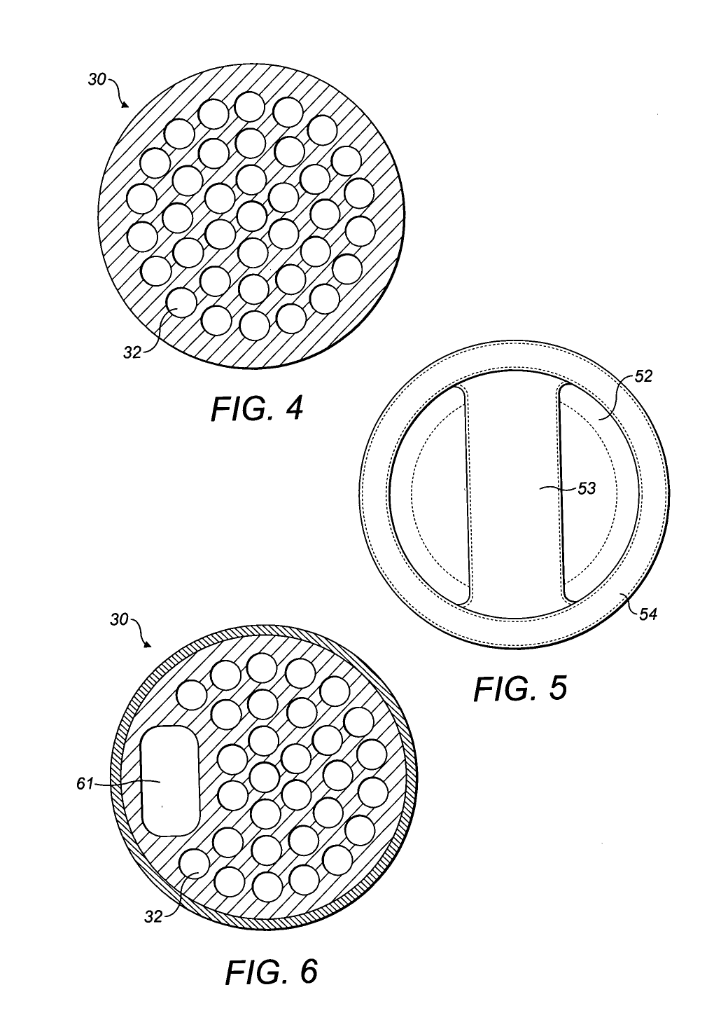

FIG. 4 is a cross-section of the container of FIG. 1 on the line A-A showing an internal barrier thereof;

FIG. 5 is a view corresponding to FIG. 3 of a container with a modified lid;

FIG. 6 is a view corresponding to FIG. 4 of a container with a modified barrier;

FIG. 7 is a view of a modified container;

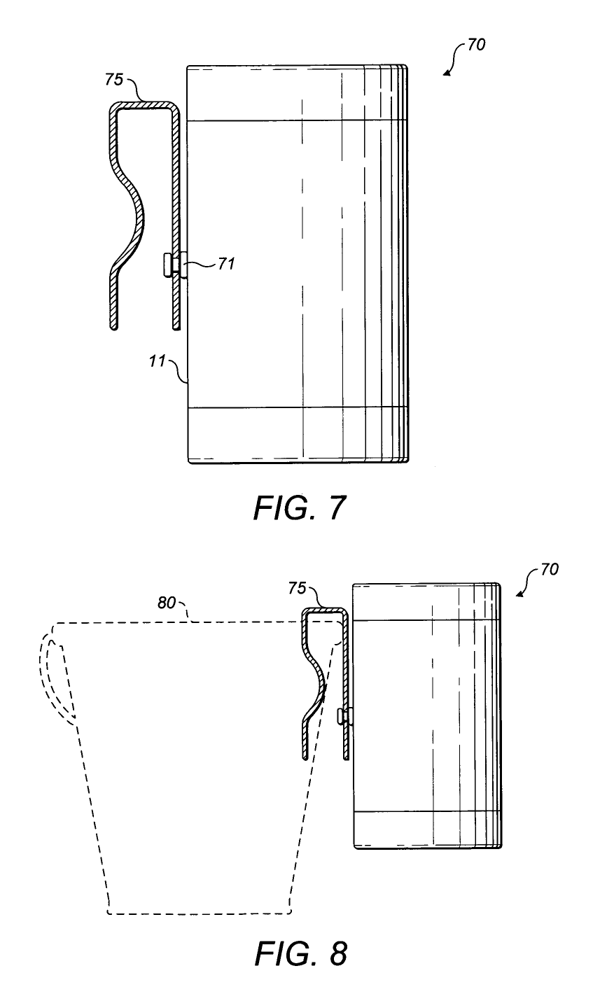

FIG. 8 is a view on a reduced scale of the container of FIG. 7 in use in combination with a bucket;

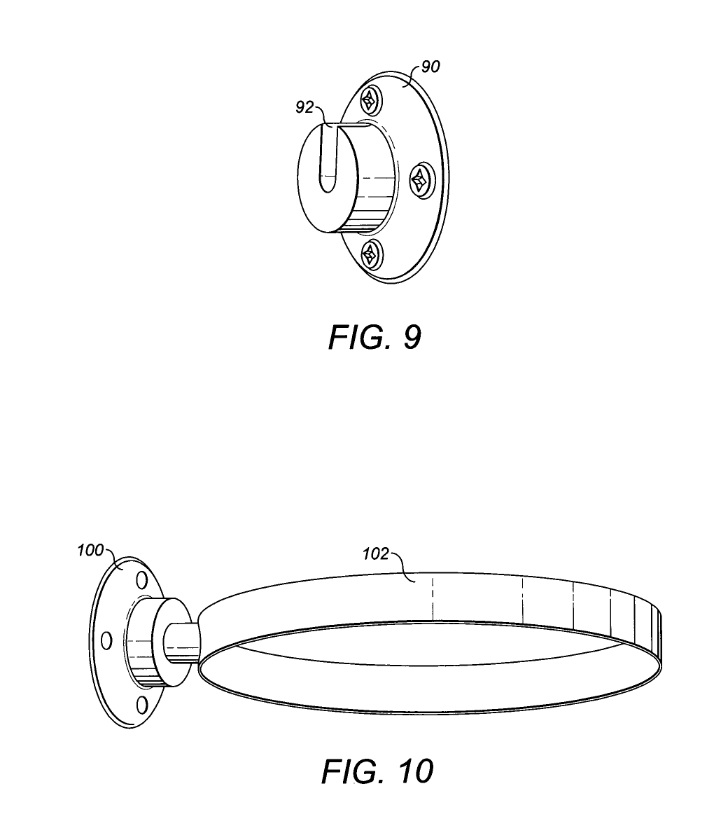

FIG. 9 is a view of a mounting for arrangements in accordance with the present invention; and

FIG. 10 is a view of a modified mounting for arrangements in accordance with the present invention.

Referring to the drawings, FIG. 1 shows a container 10 comprising a cylindrical wall 11 and end caps or lids 12 and 14 located on the ends 16 and 18 of the cylindrical wall. Wall 11 and lids 12, 14 are of an opaque moulded plastics material. Lids 12, 14 are removably attached at opposite ends of the container by respective quarter-turn connection arrangements. Each connection arrangement is liquid-tight.

As shown in FIGS. 2 and 3, each lid has a circular finger recess 21 surrounding a central projection part 22.

Moulded integrally with the cylindrical wall 11 and located midway between the ends 16, 18 thereof is a divider or barrier 30. Thus the barrier divides the container into two substantially identical spaces 36, 38. As shown in FIG. 4, barrier 30 has a plurality of holes 32 therethrough so that it is permeable to liquid. Thus the barrier is capable of serving as a filter with a plurality of drainage filter holes.

The container 10 is used to store articles such as dish cloths, sponges and/or scouring pads. Its use will be described, by way of example, with reference to dish cloths in a kitchen.

With end cap 14 tightly-closed, a relatively-mild disinfectant or sterilising solution is poured into the open end 16 of the container 10 until the liquid level in space 38 is just below the barrier 30. Conveniently, a concentrate dose in liquid, pill or capsule form can be used with water filled to an indicated level. When a user has temporarily finished using a cloth, it is inserted into the space 36 above barrier 30. Lid 12 is then locked into position and the entire container is inverted, i.e. rotated through 180° about a horizontal axis, so that lid 12 is at the bottom. The container 11 may then be shaken. It is then placed on any convenient horizontal surface. The action of inversion causes the liquid in space 38 to fall through barrier 30 into space 36 where it surrounds the cloth. The cloth is thus immersed in the liquid and is subject to a disinfecting or sterilising action.

When it is desired to re-use the cloth, the container 10 is again inverted, i.e. back into its original disposition with lid 12 at the top. The liquid then falls back from space 36 through barrier 30 into space 38. The user may wait a few moments for the liquid to complete draining. Lid 12 is removed and the user may remove excess liquid by squeezing the cloth against the top surface 37 of the barrier. The cloth may then conveniently be rinsed under a tap before further use.

The above procedure is repeated after each period of use of the cloth. At regular intervals, e.g. once per day, the solution in the container is discarded and replaced by a fresh solution. At greater intervals e.g. once per week, both end caps 12, 14 are removed to allow cleaning of the entire interior of the container, and the removal of any particles which have been retained in space 38.

Of course, since end caps 12, 14 are substantially identical, space 38 may instead be used to accommodate a cloth in use, with end cap 14 constituting the repeatedly removed lid.

The above-described arrangement has numerous advantages. Use of the container 10 provides an attractive and hygienic way of temporarily storing articles such as dish cloths, sponges and scouring pads between their periods of use. The disinfectant solution is efficiently used, since the same liquid is used many times to disinfect or sterilise articles.

Since the container is less than half full of liquid, any inserted article sits on the barrier clear of the liquid surface when the container is returned to its original disposition. This arrangement does not expose the user's hands to immersion in the disinfectant solution. When the user removes the article from the container, the bulk of the solution has already drained therefrom. Even more liquid can be removed from the article by the user pressing on the article to squeeze it out against the barrier.

By being integrally moulded with the container walls, the barrier provides a robust structure which is able to resist the pressure as the user squeezes the article against it. Moreover, the barrier 30 merges smoothly with the container wall in a continuous curve so that there are no corners in which dirt can accumulate. In addition, the barrier can not be separated from the container and lost.

By having end caps 12, 14 at both ends which can be opened, the container can be put into use either way up.

Various modifications may be made to the above-described arrangement. For example, the barrier member 30 may be closer to one end of the container 10 than the other. This can be convenient for squeezing out the cloth when the size of the user's hand makes it difficult to press effectively against a barrier which is centrally-located. A non-centrally placed barrier also has the advantage of providing differently-sized spaces 36 and 38, which are suitable for accommodating articles of differing sizes.

Depending upon the liquid used, the container 10 and/or the end caps 12, 14 may be of a transparent or semi-transparent material such as acrylic or polypropylene. The container and/or the end caps may be of other materials than plastics, e.g. of a metal or glass.

Instead of being integral with the container wall 11, the barrier 30 may be a separate member capable of being fitted to a suitable seating within the container wall. Two or more seatings may be provided at different heights on the interior of the container wall 11; thus the user may have the choice of placing the barrier 30 substantially centrally or closer to one end. Also, the barrier may be of a different material (metal) from the container (plastics). In a further modification two internal barrier members 30 are provided.

The container may have any convenient cross-sectional shape, e.g. square, rectangular, polygonal, elliptical or irregular. The cross-section may vary so that the ends are narrower or alternatively wider than the central region. A varying cross-section with a slightly narrow waist in the central region provides a container which is easy to hold. In further examples, the container may be spherical or frusto-spherical in shape.

The end caps or lids 12, 14 may be connected to the container wall by any convenient connection arrangement, e.g. by a connection involving more than a quarter-turn, by a screw thread of one or more full turns, by a bayonet type arrangement, or by a push-button type arrangement.

One end cap may be arranged to be more readily removable than the other end cap. This would be the one intended for removal during regular (daily) use while the other would be for less frequent (weekly) removal for thoroughly cleaning the entire interior of the container.

In an alternative modification, only one end cap is provided with the other end forming a closed end integrally formed with the container wall.

The lids 12, 14 can be of any suitable shape or configuration; for example the central projecting parts 22 may be square instead of circular.

A modified lid 52 is shown in FIG. 5 having a grip part in the form of a diagonal web member 53 extending across a circumferential rim 54.

Seals may be provided between the end caps and the container wall, although such an arrangement may take longer to clean.

Before removing the article from the container, the user preferably presses the article against the barrier manually. Alternatively, the pressing may be effected by a separate implement or by a lever member attached to the barrier or to the interior of the wall.

The size and arrangement of the holes 32 in the barrier may be selected as desired. They may be constituted by slots. The holes may be small enough to resemble the mesh of a net.

As shown in the modification of FIG. 6, one or more larger holes 61 may be provided in the barrier to accommodate specific articles, such as a brush. The barrier 30, or the interior of the container walls 11, may be provided with appropriate seatings for accommodating other items.

The container 10 may be provided with means for attaching it to another item such as a bucket or larger receptacle, or a draining board, work surface or sink. Alternatively the container may be manufactured integrally with such items.

The container 10 may be mounted to pivot about a horizontal axis. In a first pivotal arrangement the exterior of the wall is provided with a moulded spigot 71 FIG. 7 extending from a point midway along the height of a container 70. A substantially U-shaped clip 75 is attached to the spigot 71, with the spigot passing through a hole in the clip to permit relative rotation. The clip can readily be mounted on the edge of a bucket 80, FIG. 8, or other receptacle where the container can be rotated between its vertical dispositions.

In a modification, the container is mounted in a wall bracket 90, FIG. 9. Bracket 90 is arranged to be screwed to a wall or other vertical surface and has a recessed slot 92. In this modification clip 75 is omitted and slot 99 is arranged to directly receive spigot 71 in a manner which permits relative rotation.

In another pivotal arrangement, FIG. 10, a wall bracket 100 pivotally supports a ring 102 which is of a size to accommodate container 10. A container 10 is secured in ring 102 so that it can readily be rotated between its vertical dispositions. Means may be provided for releasing the container from the ring 102 to allow for intermittent cleaning of the various parts.

The pivotal arrangements may be attached to a kitchen wall or a cupboard door. If attached to the inside of a cupboard door, they give a kitchen a tidier appearance. Alternatively, the pivot may be mounted on a free-standing support. The pivot enables the container 10 to be quickly inverted between its two vertical dispositions. A detent means may be provided to retain the container in its vertical dispositions.

The container 10 is not restricted to employment in domestic kitchens. A larger version can be used in commercial establishments. Containers and/or end caps with different colours and/or sizes and/or different labelling can be used to store different cloths or other articles for different purposes. The colours or other distinguishing features are advantageous where different liquids or solutions of different strengths are to be employed in the different containers.

The container 10 can be alternatively used for the sterilisation of flannels and sponges in bathrooms. Containers and/or end caps of different colours could be used to avoid confusion with containers intended for kitchen use.

The container 10 can be alternatively used for medical applications, e.g. to store surface wipes and other cleaning equipment.

The container 10 can be alternatively used for preserving items, such as items of food which are to be stored under a liquid, such as vinegar, sugar syrup or oil.

When pouring liquid into the container, the liquid level may be well away from the barrier if desired, e.g. only to a depth of one quarter of the height of the container. Alternatively the liquid level may be level with or even above the barrier if required.

The container may be provided in a package or kit together with doses for making up the liquid in the container. Thus the container may be provided with a substance in concentrated liquid, gel, powder, tablet or capsule form in a separate box, sachet or bag etc. Alternatively, a supply of dosing material may be contained in a suitable refillable compartment in one or both lids. For example, if the dosing material is in tablet form, individual tablets can be dispensed by a built-in clicker mechanism similar in principle to the type used to disperse sweetener in drinks. If the dosing material is a liquid or gel form, the built-in dispensing mechanism can resemble a pump-style liquid soap dispenser.

In a further modification the or each lid incorporates an internal, permeable compartment for releasing a material to dissolve in the liquid in the container. The compartment may have a removable permeable cover.

The lid may not be refillable, in which case a new lid needs to be acquired when an old one runs out of dosing material.

The lid may have an internal wafer or other indicating means which indicates, e.g. by a colour change, when the dosing material needs replacing or refilling.

The features of the various embodiments and modifications may be combined or interchanged as desired.

1.-20. (canceled)

21. A method of temporarily storing a loose article at least partly under a sterilising liquid employing a closable container having an interior space which is divided into two by a permeable barrier, the method comprising the steps of providing the sterilising liquid in a space below the barrier, inserting an article into a space above the barrier, closing the container and then inverting the container.

22. A method according to claim 21, wherein the barrier is integral with the container.

23. A method according to claim 21, wherein the container has two ends and the barrier is attached to the container substantially halfway between its ends.

24. A method according to claim 21, comprising providing the sterilising liquid in the space below the barrier to a level which is just below the barrier.

25. A method according to claim 21, wherein the article is a cloth.

26. A method according to claim 21, wherein the barrier has a plurality of through holes.

27. A method according to claim 26, wherein at least one of the holes is larger than the other holes, and the method comprises the step of accommodating a further article in said at least one hole.

28. A method according to claim 27, wherein the further article is a brush.

29. A method according to claim 21, wherein the container is rotatably mounted on a horizontal pivot and the container is inverted by rotating it about said horizontal pivot.

30. A closeable container having an interior space which is divided into two by a permeable barrier, the space below the barrier containing a sterilising liquid.

31. A container according to claim 30, wherein the barrier is integral with the container.

32. A container according to claim 30, wherein the container has two ends and the barrier is attached to the container substantially halfway between its ends.

33. A container according to claim 30, wherein the container has a removable cap at each end.

34. A container according to claim 30, wherein the barrier has a plurality of through holes.

35. A container according to claim 30, further comprising a cloth positioned above or below the barrier.

36. A container according to claim 35, further comprising a brush, wherein at least one of the holes is larger than the other holes and wherein said brush is accommodated in said at least one hole.

37. A container according to claim 30, wherein the container is rotatably mounted on a horizontal pivot.

38. A container according to claim 30, in combination with a bucket or other receptacle, wherein the container is attached to the side of a bucket or other receptacle.

39. A container according to claim 30, in combination with a kitchen unit, wherein the container is attached to a drawing board, cupboard door or work surface of the kitchen unit.

40. A closable container comprising a removable cap for opening and closing the container at one or both ends, the container having an interior space which is divided into two by an integral permeable barrier spaced from both ends, wherein the container in rotatably mounted on a substantially horizontal pivot.
